(19) United States
(12) Patent Application Publication
(10) Pub. No.: US 2022/0296355 A1
(43) Pub. Date: Sep. 22, 2022

(54) DEVICE AND METHOD FOR URETHRAL APPLICATION OF A LUBRICATING AND/OR LUMEN-STRAIGHTENING SUBSTANCE THROUGH A RETRACTABLE INSERTION AID

(71) Applicant: Creative Balloons GmbH, Waghausel (DE)

(72) Inventor: Fred Göbel, Speyer (DE)

(21) Appl. No.: 17/636,210

(22) PCT Filed: Sep. 4, 2020
(86) PCT No.: PCT/IB2020/058241

§ 371 Date: Feb. 17, 2022

(30) Foreign Application Priority Data
Sep. 4, 2019 (DE) 10 2019 006 229.9

(51) Int. Cl. A61F 2/04; A61B 46/23; A61B 46/00; A61M 25/00; A61M 25/01; A61F 5/44

(52) U.S. Cl. CPC A61F 2/04; A61B 46/23; A61B 46/30; A61M 25/0017; A61M 25/0102; A61F 5/4405; A61B 2046/205

(57) ABSTRACT

The invention relates to a device for draining urine from the bladder of a patient, consisting of an indwelling part placed in the bladder and in the urethra, and of a retractable insertion aid of tubular or hose-like construction which is placed into the indwelling part and facilitates insertion into or passage through the urethra, and of which the inner lumen affords the possibility of applying a lubricating and lumen-straightening substance into the urethra, wherein a reservoir vessel for holding a preferably gel-like substance can be mounted at the proximal end of the insertion aid, and the substance can be applied through the insertion aid from the opening of the catheter tip into the urethra, without a reverse flow into the proximal gap between insertion aid and catheter; moreover, various embodiments of the device are described, and also a method which permits the procedure of draping and cleaning in preparation for the introduction of a catheter by hand and also allows the catheterization of the bladder to be guided with just one hand by the user.

### REFERENCE TO PENDING PRIOR PATENT APPLICATIONS

This patent application claims benefit of International (PCT) Patent Application No. PCT/IB2020/058241, filed 4 Sep. 2020 by Creative Balloons GmbH for DEVICE AND METHOD FOR URETHRAL APPLICATION OF A LUBRICATING AND/OR LUMEN-STRAIGHTENING SUBSTANCE THROUGH A RETRACTABLE INSERTION AID, which patent application, in turn, claims benefit of German Patent Application No. DE 10 2019 006 229.9, filed 4 Sep. 2019.

The two (2) above-identified patent applications are hereby incorporated herein by reference.

### FIELD OF THE INVENTION

The invention addresses a device and a method for draining urine from the bladder of a patient.

### BACKGROUND OF THE INVENTION

Urinary catheters for continuous, closed draining of urine from the bladder of a patient in in a collection vessel are established in medical practice and various embodiments thereof are known. Indwelling urinary catheters are normally introduced into the urinary bladder by transurethral catheterization. In contrast to catheterization of the bladder with disposable catheters, during which a catheter-tike tube is advanced intermittently through the urethra into the interior of the bladder, i.e., only for the actual duration of emptying the bladder, catheters for permanent transurethral draining of urine are equipped at their distal end typically with a balloon-like anchor element (retention balloon), the primary function of which is securing the position of the catheter end that receives the urine within the bladder.

In the case of the overwhelming majority of known design types of so-called indwelling catheters, these retention balloons are made of elastically expandable tube materials, which are nestled snuggly against the shaft body of the catheter and are seated as much as possible without any preformation as smooth, fold-free, cylindrically formed tube bodies (sleeves). Because they are filled with a non-compressible fluid medium, an approximately spherically formed balloon body expands from the cylindrical tube body when there is a correspondingly high filling pressure. What is disadvantageous in the case of these types of anchor balloons that are expanded by high filling pressure is above all the hard consistency of the balloon resulting from the expansion of the sheath. In the case of a longer indwelling duration, erosive changes and to some extend also pronounced ulcerations of the bladder's mucous membranes occur in the contact area of these types of rigid, “hard” balloons on the base of the bladder, in particular in the region of the so-called bladder trigonum. In the case of the sustained presence of bacteria in the urine, chronic infections of the urinary tract can develop based of these types of tissue defects. The current research in the field of urinary tract infections (UTIs) values an epithelium of the bladder and also of the urethra that is as intact as possible and as non-eroded as possible as an essential prerequisite for preventing chronic urinary tract infections.

In addition to conventional, sleeve-based catheters, retention elements based on preformed balloon components are also known from the literature. These kinds of components are presented for example in EP1448259 or PCT/IB2015/002282. The retention in this case is guaranteed by balloon components that are formed completely or even partially in terms of the working dimensions required for the respective function already during manufacture of the catheter. The balloon elements are made of membranous thin-walled, but high-strength materials, which in an unfilled state are minimally applied to the catheter shaft in such a way that the formed balloon component can be advanced comfortably and without irritation through the urethra during insertion of the catheter.

More recent literature on the subject of UTIs furthermore makes reference in many cases to the traumatizing effect of conventional catheter tips on the dome of the bladder that is exposed to the tip. The normally relatively rigid tip, which extends beyond the retention balloon and finger-like into the bladder lumen, burrows into the tissue of the dome of the bladder with sustained exposure and thus leads regularly to other lesions that are directly associated with catheters.

Embodiments of catheter tips in a variety of designs are known that prevent lesions of the urethral wall, in particular during insertion or advancement of the catheter through the urethra, and are meant to facilitate catheter placement as a whole. Several design types moreover attempt to guard against injuries to the dome of the bladder. For example, dual balloon arrangements are described, such as is the case with the Duette type from Poiesis Medical Ltd., wherein the tip of the catheter is accommodated in a protective manner in a spherical, balloon-like element, while another balloon element rests in a retaining manner on the base of the bladder. In the case of Duette, the urine is drained through an opening of the catheter shaft between the two balloons.

Another effect that directly traumatizes the urinary tract is caused by the structure of the urine-draining shaft segment of the conventional catheter. The shaft attaches proximally at the balloon being retained in the bladder and drains the urine transurethrally. The shaft element is typically made of a tube material that is designed to be relatively thick-walled and is based on silicone, or is made of materials based on natural rubber. The thick-walled design of the shaft wall ensures, on the one hand, that the urine-draining lumen does not get occluded from kinking in the case of axial bending or twisting of the shaft. On the other hand, the thick-walled shaft structure ensures that the urine-draining lumen of the shaft does not collapse from the high pressures acting in the balloon during the elastic expansion of the balloon sleeve into a spherical balloon. In particular, the shaft wall thicknesses required by the balloon filling pressure restrict the structurally achievable diameter of the urine-draining lumen. Draining through small diameters tends to lead to premature encrustation or successively to occlusion from the ongoing deposition of urine components. Therefore, in order to achieve the longest possible application periods, the largest possible inner diameters are striven for, which require, in turn, large shaft outer diameters. The approximation of the shaft outer diameter to the diameter of the urethra that is consequently required in the case of a conventional catheter design leads, in conjunction with the rigidity of the shaft materials, frequently to pronounced erosions of the epithelial lining of the urethra, whereby, in the case of a long indwelling duration, this regularly results in inflammatory processes, and subsequently in cicatricial strictures of the urethra.

Another factor that supports the development of chronic urinary tract infections is the accumulation of so-called residual urine in the bladder. Residual urine empties out of the bladder only in delayed manner and therefore is apt to accumulate larger amounts of bacteria. Various embodiments are known which prevent the pooling of urine, predominantly by draining the urine via openings of the catheter shaft, which are positioned especially close to the base of the bladder.

### SUMMARY OF THE INVENTION

In order to prevent injuries during the placement of the catheter, gel-like substances are being used with good success. On the one hand, they reduce the friction between the catheter shaft surface and the epithelium of the urethra. Beyond this lubricating effect, gels make a transient straightening or filling the urethra lumen possible. In a more or less circularly straightened state of the urethral lumen, the catheter tip and the catheter shaft can thus be inserted in an optimum manner with low friction through the lumen of the urethra.

Urinary catheters are frequently packed in a set containing all paraphernalia required for the catheter placement. The ready-to-use sets are generally speaking unnecessarily extensive, and in many cases no longer self-explanatory in terms of handling and hinder the application of the product.

The object of the invention is to avoid the disadvantages of the prior art described in the foregoing.

To address this problem the invention provides, as a part of a device for draining urine from the bladder of a patient, a functional unit comprising
a) a shaft-shaped indwelling part that can be placed in the bladder and in the urethra that drains in the manner of a catheter and has at least one drainage opening in the region of the distal tip of the shaft-shaped indwelling part; and
b) a retractable insertion aid that is inserted into the shaft-shaped indwelling part, which facilitates insertion into and passage through the urethra, and has a tubular or hose-like construction with an apical opening for terminal release of a lubricating and/or lumen-straightening substance through the opening.

As a part of the present patent application, the terms “indwelling part”, “indwelling unit”, “catheter” and “urinary catheter” are used synonymously to a great extent.

The present invention describes in particular the application-optimizing connection of a urinary catheter to a removable insertion element inserted into the urine-draining lumen of the catheter, which insertion element stabilizes the catheter shaft for the urethral insertion and allows the user-friendly intra-urethral insertion of lubricating or lumen-straightening gel. The simple handling of the catheter is provided for within the scope of the invention by corresponding structural modifications and ancillary additions. In particular, the invention offers the possibility of a catheterization guided by one single hand, which in an optimal case includes the lubrication of the catheter tip, the insertion of the front third of the catheter, the lumen-straightening of the urethra through the application of gel, the successive pushing forward of the catheter with a tong-like gripper element, and the blocking of the retention balloon. The special design of the device according to the invention allows the scope of a catheter set to be kept to a minimum.

The invention furthermore describes the especially atraumatic design of all components of a urinary catheter that are placed intracorporally. In particular, the catheter shaft is supposed to be as tissue-friendly as possible, and behave in a way that avoids erosions in the urethra. Perforating injuries to the dome of the bladder from the tip of the catheter are supposed to be prevented. The catheter balloon should have the best possible retaining and nestling sealing effect in the region of the urinary outlet, wherein an eroding effect of the balloon wall on the epithelium is as low as possible. The lumen of the catheter shaft draining the urine is supposed be optimally large. Moreover, the pooling of residual urine in the bladder is supposed to be minimized.

The invention describes in particular a combination of a shaft tube, which is indwelling in an atraumatic manner in the urethra, designed to be thin-walled and/or have a large lumen, and drain urine effectively, with an insertion aid inserted into the thin-walled shaft tube, which insertion aid facilitates the release near the tip of a lubricating and/or lumen-straightening substance into the urethra along with the stabilizing reinforcement of the catheter shaft that is required for catheter placement. After securing the catheter by filling the catheter balloon, the insertion aid is pulled out of the catheter lumen so that a most malleable, friction-minimizing tube body possible comes to lie in the urethra, which prevents the typical, erosion-induced lesions of the urethra in the case of conventional catheterization.

In the preferred construction as a hose-like or tubular body, the insertion aid comprises an inner lumen measuring approx. 1.0 to 3.0 mm, through which a lubricating and/or lumen-straightening substance is released in the region of the tip of the urinary catheter accommodating the insertion aid. In the process, the respective substance exits as close as possible to the urine-draining opening of the urinary catheter spaced apart in the draining lumen of the catheter. As an alternative, the tip of the insertion aid can reach in an axial extension of the catheter portion indwelling in the bladder, beyond an outlet of the catheter shaft opening towards the front. With such a design, the insertion aid can be provided in the region of its tip with one of the many tip formations that are common in transurethral catheterization, and which facilitate the passage of the catheter through critical bends in the progression of the urethra. In this case as well, the insertion element comprises an inner lumen for the application of a lubricating and/or lumen-straightening substance, wherein the release in this case takes place directly from an opening of the insertion element into the urethra. Following the securing of the catheter in the bladder, the insertion aid is removed. After the insertion aid is retracted, in the preferred case, a funnel-like outlet adjusts in the front balloon radius of the catheter balloon, wherein the opening of the catheter shaft is spontaneously positioned at the base of the funnel during the filling of the balloon.

Alternatively, the catheter shaft can comprise a short, spout-like tip formation, which, however, is designed in such a way that it does not project beyond the front radius of the balloon in a balloon that has been placed in situ.

Furthermore, the invention describes a special formation of the catheter tip, which comprises a corrugated-tube-like wall formed to be corrugated, which makes the tip freely rotatable or bendable in every direction, and thereby, on the one hand, simplifies the passage through the urethra, and, on the other hand, allows the tip to be bent axially on all sides within the bladder, so that the dome of the bladder does not get irritated and damaged by a formation pointing sharply at it when it is resting on the distal end of the catheter.

The balloon body is preferably formed as flat or discoid as possible in the case of all embodiments of the device according to the invention in order to reduce the formation of residual urine.

In line with an optimally simplified handling during the catheterization, the lubricating and/or lumen-straightening substance can be injected in a first handling step into a hood-like or bag-like sheath, which is seated on the distal end of the catheter or the distal end of the insertion aid with a narrow, intermediate gap space and which distributes the substance as uniformly as possible in this gap space around the catheter tip or the distal end of the catheter. The sheath is then removed towards the front immediately before insertion of the catheter into the urethra.

The reinforced catheter that is stabilized by the insertion aid is inserted approximately 8 to 10 cm into the male urethra in a manner that is guided in a quasi piercing manner. In order to facilitate the further passage of the catheter in an optimally atraumatic manner, following the insertion of the front catheter section, a second gel quantity is injected into the urethra which straightens the lumen of the organ and thus prevents injuries in particular there where the male urethra takes an especially curve-like winding course.

The occlusion of the drainage lumen from deposits of urine components is counteracted in the course of the invention by a maximally large-lumen design of the urine-draining lumen. For this purpose, the wall of the transurethral segment of the catheter is reduced as far as possible, in an extreme case to the film thickness. In order to prevent lumen-occluding kinks or twisting of such a thin-walled shaft, said shaft can be provided with a lumen-stabilizing, corrugated-tube-like profile over the entire length, or even in only specific, mechanically relevant sections. In conjunction with materials having a high elastic restoring force, such as e.g., polyurethane, a shaft with a such a profile can be provided with spontaneously lumen-straightening properties, which facilitate namely an elastically acting, malleable bending of the shaft, as well as ensure an easy to effectuate transient, radial collapse of the lumen, but, on the other hand, a reliable, spontaneously lumen-straightening and disentangling effect.

In addition, in combination with tubular-film-like, thin-walled shaft walls, for example rod-like, hose-like, tubular-mesh-like structures are also conceivable, which run freely in the interior of such a drainage tube in a torsion-inhibiting, stabilizing manner.

The invention uses preferably specially formed balloon components, which are described for example in PCT/IB2015/002282. These balloons manufactured from micro-thin-walled, but simultaneously highly dimensionally-stable flexible film are preferably filled incompletely, only flaccidly with air. Due to the placement in a flaccid, unexpanded state, abrasions of the epithelium lining the bladder that have a traumatic effect can be prevented to a large extent. In the case of an unexpanded, flaccid filling of the balloon, only the respective force acting in the bladder will be absorbed by the balloon. The filling pressures are thereby in a low millibar range. In an unloaded state of the balloon, they do not or only slightly exceed the intravesical pressure prevailing in the bladder or the patient.

As described in PCT/IB2015/002282, a balloon of this type can also be provided with a urethral extension formed cylindrically and with a tapered diameter. The retaining, vesical balloon portion in this case merges proximally into a sealing, urethral balloon portion. The combined, vesical-urethral balloon body is provided in the urethral segment preferably with a “residual” diameter, which exceeds the diameter of the lumen of the urethra by approximately 0.5 to 1.5 times, preferably by 0.5 to 1.0 times. The balloon portion whose diameter is over-dimensioned in this way is thereby applied to the urethral wall in a tension-free manner that is largely independent of size, under the formation of a radially oriented fold or invagination of the sheath wall. The residual excess ensures that all portions of the urethra can be sealed in a flaccidly tamponaded manner. In addition, diameter fluctuations of the urethral lumen can be balanced out through the residual diameter.

As an alternative, less preferred design, the urethral portion of the formed balloon body can be dimensioned in such a way that it essential corresponds to or exceeds the diameters of the respective urethra.

The filling of the catheter balloon used according to the invention takes place preferably with a gaseous medium or with air. The supply of the filling medium takes place via a supply line integrated into the shaft wall, but can also take place through supply lines that run freely in the shaft lumen.

The invention also takes less preferred designs into consideration that dispense with the described, preformed balloon components and instead comprise conventional, retention components that stretch elastically from a tube section.

The indwelling part and the insertion aid can be made of materials which have the same hardness. However, design variants are preferred in which the materials for the indwelling part and for the insertion aid differ from each other at least in terms of their hardness.

Because both parts are made of plastics, preferably of thermoplastics or thermosetting plastics or elastomers, the material hardness is preferably measured as Shore hardness. The hardness values in this case are especially preferably Shore A or Shore D.

The Shore hardness y of the material of the insertion aid should correspond or exceed that of the Shore hardness x of the material of the indwelling part:

y=x

or

y>x

or

y≥x.

The difference in the Shore hardnesses, in particular the hardnesses according to Shore A, should be defined as follows:

y−x≥0

or

y−x≥1

or

y−x≥2

or

y−x≥5

or

y−x≥10

or

y−x≥20.

A method according to the invention for the catheterization of the bladder of a patient is characterized in that a lubricating and/or lumen-straightening substance is released from a compressible reservoir vessel as a result of a digital or manual compression of the reservoir vessel.

In the process, a prepared, ready-to-use catheter device is used, which is made of a shaft-shaped indwelling part that can be placed in the bladder and in the urethra that drains in the manner of a catheter and has a drainage opening in the region of the distal tip of the shaft, and a retractable insertion aid that is inserted into the shaft-shaped indwelling part, which facilitates insertion into and passage through the urethra, and has a tubular or hose-like construction with an apical opening for terminal release of the lubricating and/or lumen-straightening substance, on whose proximal end the reservoir vessel is connected to or can be connected to the lubricating and/or lumen-straightening substance.

### BRIEF DESCRIPTION OF THE DRAWINGS

Various embodiments of the urinary catheter device according to the invention are described in the enclosed figures, along with combinations with special, functional accessory components for carrying out the catheterization with one hand. They show:

FIG. 1 An embodiment in which a substance-applying insertion element that stabilizes the catheter shaft is inserted into the urine-draining lumen of the urinary catheter.

FIG. 2 An embodiment in which a substance-applying or stabilizing insertion element is inserted into the urine-draining lumen of the urinary catheter in such a way that the tip of the insertion element reaches beyond the distal, terminal opening of the catheter shaft that is indwelling in the body.

FIG. 2a A special design of the tip of the insertion element that is formed with a corrugated profile.

FIG. 2b A corresponding design of the distal end of the catheter that is indwelling in the body and is formed in a corrugated manner.

FIG. 2c An insertion element formed of a single piece and having an integrated, terminal compressible substance reservoir.

FIG. 2d A special design of an insertion element, which comprises a terminal, funnel-like receiving piece for the insertion and perforating opening of a structurally separate reservoir vessel.

FIG. 3a A special design of the catheter shaft formed in a corrugated manner in sections or over the entire catheter shaft.

FIG. 3b A further embodiment of the catheter shaft, wherein said shaft comprises an especially thin-walled, tubular-film-like nature.

FIG. 4A reservoir vessel that can be compressed in a locking manner for lubricating and/or lumen-straightening substances.

FIG. 5A special arrangement of a hood-like element for the lubrication of the catheter tip.

FIG. 6A filling device that can compress in a bellows-like manner for filling the catheter balloon.

FIG. 7A holding device for the U-shaped looped accommodation of a catheter for the optimized, ready-to-use insertion of the catheter with only one hand.

FIG. 7a A special tweezer or tong mechanism for gripping the catheter shaft; and

FIG. 8A special embodiment of a two-layer surgical drape, which the user can pick up in a glove-like manner and guide to the opening to be catheterized.

### DETAILED DESCRIPTION OF THE PREFERRED EMBODIMENTS

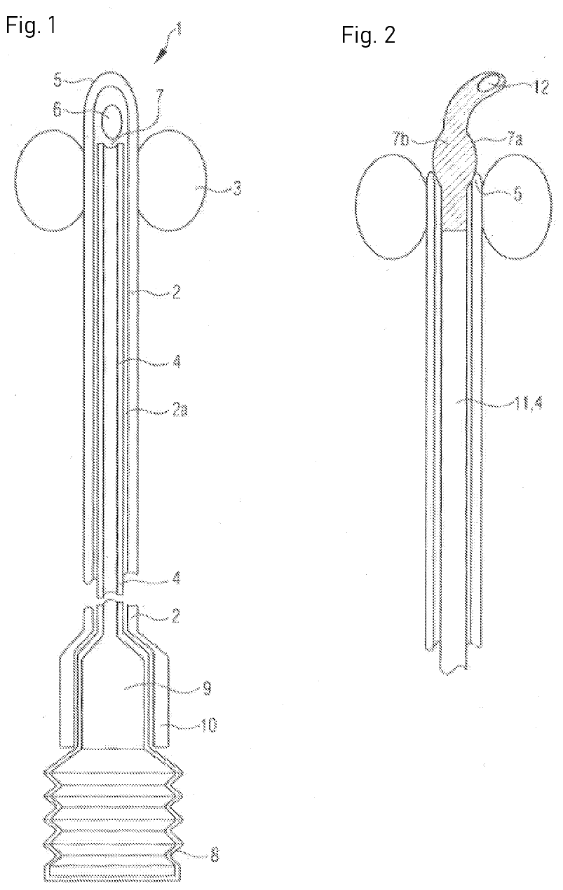

FIG. 1 depicts a variation of the embodiment of the catheter device 1, in which the catheter shaft 2 of the device is equipped with a balloon 3 that is already formed during manufacturing with the working dimensions required for the retention of the catheter in the bladder, wherein the balloon is preferably manufactured of a thin-walled polyurethane film whose volume is only a little expandable. A hose-like or tubular insertion aid 4 is inserted into the urine-draining lumen 2a of the catheter shaft. The tip 5 of the catheter shaft comprises at least one drainage opening 6. Preferably, two or more drainage openings 6 can also be provided, which are arranged in a distributed manner preferably around respectively equal intermediate angles over the circumference of the catheter shaft 2. For example, two drainage openings could be diametrically opposite from each other, i.e., offset by 180°, while three drainage openings would be offset from each other by 120°. In a completely inserted state, the tip of the insertion aid or its distal opening 7 comes to lie directly proximally from the drainage opening or in direct coverage with the drainage opening. The tip of the insertion aid preferably ends with the inner wall of the drainage lumen in such a way that it has a custom-fit, for example in a conically sealing or locking in a sealing manner, that the substance to be supplied during the application or injection cannot escape through the lumen of the insertion aid in a retrograde manner in the proximal part of the drainage lumen of the catheter or in the proximally adjacent gap space between the insertion aid and the inner wall of the catheter.

Located at the proximal end of the insertion aid in the preferred design is a reservoir-like, preferably folding-bellows-like reservoir vessel 8, which accommodates the substance to be supplied and from which the substance is preferably conveyed by expressing or squeezing the vessel towards the tip of the catheter. In its preferred design, in the case of the effect of an axially compressing or alternatively even a radially squeezing force, the vessel transitions into an axially or radially locking state, which prevents an elastic reestablishing of the vessel when the acting force diminishes. An aspiration of the already applied gel back into the vessel can thus be prevented.

For an improved fastening of the relative position of the catheter and insertion aid, the distal part of the vessel 8 has a cone-like segment 9, which can be inserted into the connector 10 of the catheter with a custom-fit and in an optionally locking manner.

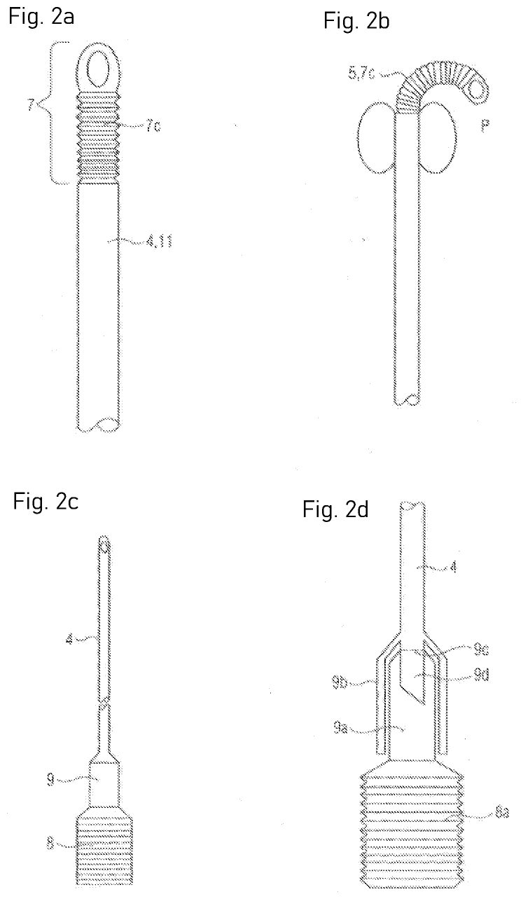

FIG. 2 shows a design of an insertion aid 4, having a molded or formed-on tip formation 7a, wherein said tip formation can be made for example of a flexible, plastisol-like material and is terminally seated on the shaft 11 of the insertion, which shaft is harder relative to the tip, for the most atraumatic possible passage through the urethra. The tip 7a preferably comprises a terminally discharging lumen 12, through which a lubricating substance can be released into the urethra in an extension of the shaft 11 of the insertion aid that is likewise provided with a lumen.

The tip 7a of the insertion aid can comprise, in the transition to the distal end 5 of the catheter shaft, a special bulge-like thickening 7b, which slims down elastically under axial tensile force, and thus, during retraction, slides out of the relative position in the transition to the catheter tip. The bulge 7b can be provided optionally with longitudinal, strip-like weakenings, which when overcoming a specific tensile force lead to the collapse of the bulge and thus facilitate the retraction of the bulge-like formation through the lumen of the catheter. The bulge 7b forms in conjunction with the front opening 5 of the catheter an atraumatic transition that is as stepless as possible. Furthermore, the bulge makes it possible for the substance, which is applied into the urethra during catheter placement, to accumulate in front of the distal catheter end 5 and be able to be pushed along in front of the catheter end so that it remains available in an adequate quantity during the urethral passage of the catheter in the region in front of the catheter tip. Particularly during the application of gels, an optimally lumen-straightening effect can be achieved when inserting the catheter into the urethra.

FIG. 2a. When using harder materials or even materials with a higher elastic restoring force, the tip 7 of the insertion aid 4 can also be provided with a profile 7c formed to be corrugated, which makes the tip flexible and axially bendable on all sides, but maintains the lumen 11 of the insertion aid. During the catheterization through the urethra, especially advantageous atraumatic insertion properties can be achieved due to the corrugated profiling.

FIG. 2b. A corresponding corrugated profile 7c can be formed also in the region of the catheter tip 5 of the urinary catheter shaft. Placed in situ, the tip can bend into an laterally bent position P, whereby a direct irritation acting in a piercing manner and, during long-term placement, an ulcerating lesion of the dome of the bladder where the tip rests can be prevented.

FIG. 2c shows a special design of the insertion aid 4, in which all functional segments, including the substance-accommodating vessel 8 at the proximal end, are formed of a single blank, preferably by blow molding from a tubular basic body. The distal portion of the insertion aid can optionally be provided with a corrugation that supports the axial bending. Furthermore, proximal shaft portions of the insertion aid can also be provided with a corresponding profile shaped in a corrugated manner in certain sections that are advantageous for facilitating passage through the urethra. In addition, the insertion aid can be provided in the proximal segment with a segment 9 formed to have a congruently custom-fit that supports the position-securing fit in the urinary catheter connector 10. As an alternative to complete blow molding from a single blank, the insertion aid according to the invention can be manufactured partially or completely also with injection molding, casting or even using dipping methods.

The insertion aid is made preferably of a material having good sliding properties, which is crucial for the comfortable retraction from the indwelling part of the device. To achieve this sliding ability, the base material used can be provided for example with a wax additive, which develops a film on the surface that reduces the frictional resistance.

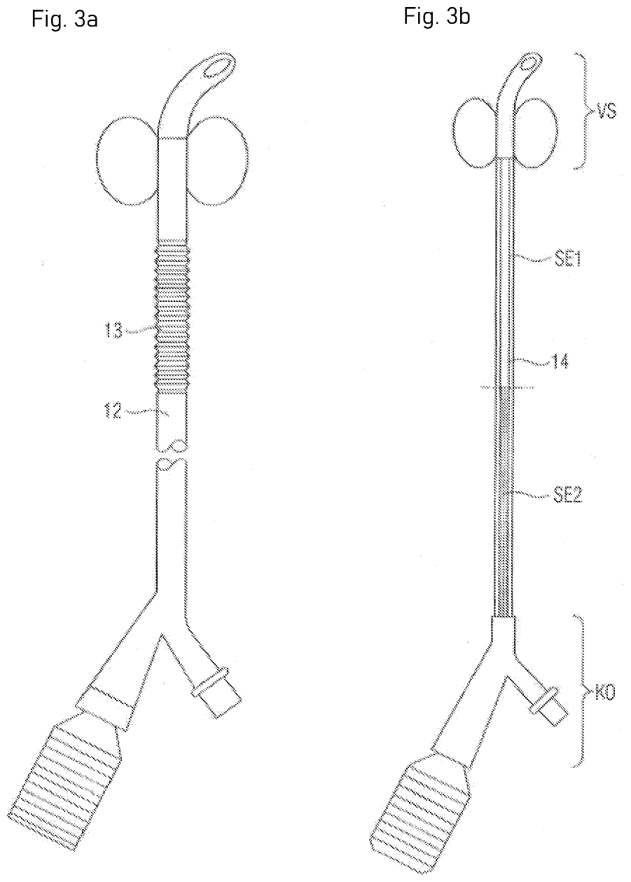

FIG. 2d shows a modified design of the design type presented in FIG. 2c, wherein the reservoir vessel 8a constitutes a self-contained unit that is not connected structurally to the insertion aid 4, which comprises a connector-like prolongation 9a, which is inserted with a custom-fit into a receiving funnel 9b with a congruent shape. The apical end of the vessel comprises an especially thin-walled or even otherwise preformed surface 9c, which is perforated when inserting the vessel, for example by a thorn-like extension 9d of the shaft portion, which extends into the receiving funnel, and thus allows the transfer of substance into the inserted combination of the catheter and the insertion aid that is prepared for the application. The design is necessary in particular for substances that require special sterilization methods that deviate from the sterilization of the actual catheter set, as may be the case for example for gel-like compounds. The vessel can therefore be enclosed separately with the catheter set and be inserted only directly before the catheterization into the end piece of the insertion aid. FIG. 3a shows a special design of the transurethral shaft segment 12 of the catheter. To guarantee a drainage diameter in the interior of the shaft that is large as possible, the shaft wall is designed preferably of a thin-walled PUR, PVC or even PVC/PUR blend material. In order to stabilize the thin-walled shaft structures and prevent a potential lumen occlusion from kinking or torsion of the shaft, the invention proposes a corrugated profile 13 of the shaft wall in the manner of a corrugated tube. The wavy corrugation of the shaft makes the respective shaft bendable and reduces potential elastic restoring effects, which are caused by the bending, or forces acting permanently on the exposed tissue from these restoring effects. Especially when using PUR, the achievable kink-free bending radius of the catheter shaft is restricted without corresponding profiling of the shaft. In addition, in contrast to PVC, PUR has a considerably higher elastic restoring force in the case of bending. The described corrugation 13 of the catheter shaft can be applied continuously over the entire length thereof or even be applied only in specified regions, in which when placed in situ an especially small bending radius is supposed to be adjusted with correspondingly low elastic tension.

For example, the shaft formed in a corrugated manner can comprise the following features: outer diameter of 6.0 mm, inner diameter of 5.4 to 5.9 mm, preferably 5.6 to 5.8 mm, polyurethane of Shore hardness 85A or 90A, corrugation amplitude of 1.0 to 1.5 mm, and peak-to-peak distance of the corrugation of 0.5 to 1.5 mm.

FIG. 3b shows a transurethral shaft tube 14 of the indwelling unit 2, which is designed to have an especially thin wall, whose wall is reduced approximately to a film thickness of 0.05 mm for example. One advantage that can be achieved with this is that such a thin shaft tube 14 is able to adapt optimally to the progression of the urethra so that even in the case of an longer application period of one or several weeks, for example, no injuries to the inner wall of the urethra are to be feared.

In order to prevent lumen occluding kinks and torsions of the tubular film 14, this is supplemented for example with a preferably free internal, rod-like, tubular or hose-like element SE1, which is depicted at the top in FIG. 3b and connects the intravesical segment VS of the catheter over the entire length of the urethra to the extracorporal connector part KO in a correspondingly stabilizing manner. In the case of a hose-like design of element SE1, the lumen thereof can be used for filling the retaining balloon.

On the other hand, in order to stabilize a tubular-film-like, thin-walled shaft tube 14, the wall thereof can also be reinforced by a net-like structure SE2 for example, which is preferably applied to the inner surface of the shaft 14 in a planar and permanently connected manner. A corresponding net-like mesh SE2 can also be permanently installed between two coaxially arranged tubular film layers, for example be coextruded with said layers. Instead of a net-like mesh, a spiroidal arrangement can also be used, or another planar arrangement with adequate self-straightening properties.

Precisely these self-straightening properties impart the otherwise very flexible tubular film 14 with the ability to resist tendencies to kink and/or reduce or reset any torsions of the tubular film 14 that occur. Both measures serve to keep the lumen open inside the film-like tubular film 14.

FIG. 4 shows a reservoir-like vessel 8, which allows the release of a lubricant into the urethra in an easily dosable manner. The vessel is preferably designed as a folding-bellows-like vessel, which, when the bellows is pushed together via pressure on the base of the 15 of the vessel engages in the respective inserted position, i.e., does not reset via an elastic effect into its erect initial dimension in the case of release of the bellows. The vessel can bear colored regions 16, which correspond to specific quantities of the substance to be applied. If the marked segments are compressed or collapsed in total, the corresponding volume in the marked segments is released.

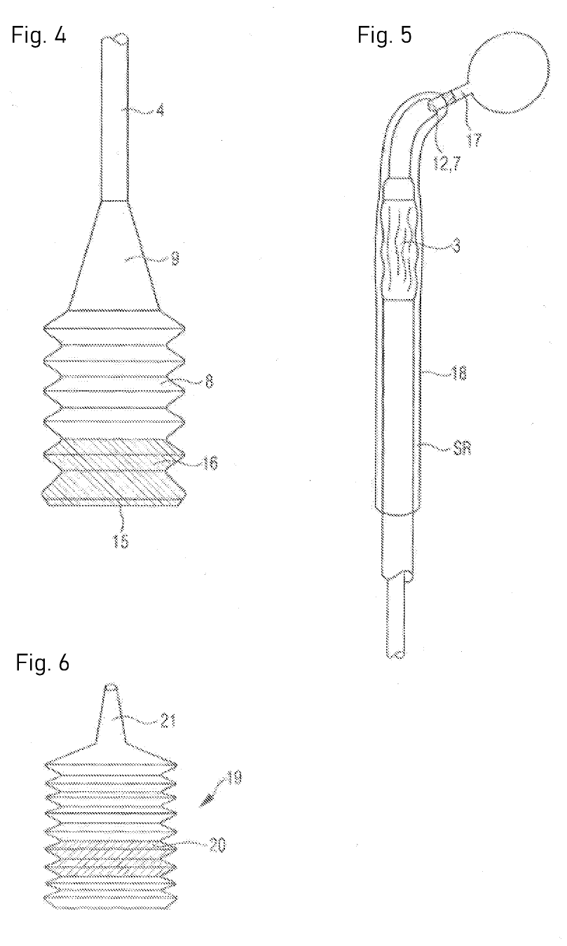

FIG. 5 shows the front section of a urinary catheter with an insertion aid 4 positioned therein for the application of a lubricating and/or lumen-straightening substance. In order to prevent the substance stored in the insertion aid from drying out, the front outlet opening 12 of the insertion element is therefore provided with a closure element 17, which hermetically seals the opening 12 of the insertion aid in a sealing manner. The lower part of the element 17 closing the opening is surrounded by a bag-like or finger-cot-like film sheath 18, which surrounds the catheter shaft with a specific gap space SR. The film sheath comprises preferably a length of approx. 8 to 12 cm. If the closure 17 is removed from the outlet opening of the front opening of the insertion aid, gel can be injected into the gap space via compression of the reservoir vessel, wherein it is distributed uniformly there. The quantity of gel required for the filling of the gap space can be specified for example by a corresponding colored marking of segments of the reservoir vessel. The user collapses the marked segment using axially acting thumb pressure on the base of the bellows, and thus conveys the required volume into the gap space.

Along with a colored marking of folding bellows segments, differently designed sections of the reservoir vessels, in particular differently configured segments of a folding bellows, can make a tactilely detectable or oriented partial quantity release possible. For example, the proximal bellows folds directly adjacent to the base of the bellows can be designed to be more smoothly compressible or compressible with lower resistance in relation to the distal bellows folds, so that, after an initial compression of the folding bellows for releasing a first partial quantity of the lubricating and/or lumen-straightening substance from the proximal segment of the bellows, the user perceives a palpable increase in resistance when transitioning to the distally adjacent folding bellows segment, which signals to him or her that the first partial quantity has been released, and the distal end of the catheter device should first be inserted into the urethra before releasing the second partial quantity of the lubricating and/or lumen-straightening substance.

Immediately before inserting the catheter into the urethra, the hood is then pulled off towards the front of the catheter by means of a closure element fastened apically to it. To prevent the film hood from being inserted unintentionally into the ostium, the hood is preferably provided apically with an approximately thumb-sized prolongation that is leaf-like for example, which furthermore facilitates the removal or the pulling off of the cap.

FIG. 6 shows an optional, folding-bellows-like vessel 19 for filling the retention balloon with preferably air, wherein the folding bellows likewise preferably has a locking fold, which ensures that the bellows 19 does not straighten elastically in the case of release and thus again suctions the filling medium from the retention balloon. To dimension the air quantity, the bellows 19 can be provided with colored markings 20, which correspondingly predefine the filling level of the retention balloon to be adjusted so that said balloon can be filled for example partially with approx. 80% of its free balloon volume, indicate a complete filling, or even produce a filling, which transfers the balloon into a moderately loaded, expanded state. In doing so, the urinary catheter is equipped correspondingly with a closure or valve element for filling the retention balloon, which following the filling, permits a self-sealing decoupling of the vessel from the catheter. The connection cone 21 of the vessel is preferably designed in such a way that, in an attached state, the cone opens the valve and allows a filling. If the cone is pulled out of the valve, the valve closes.

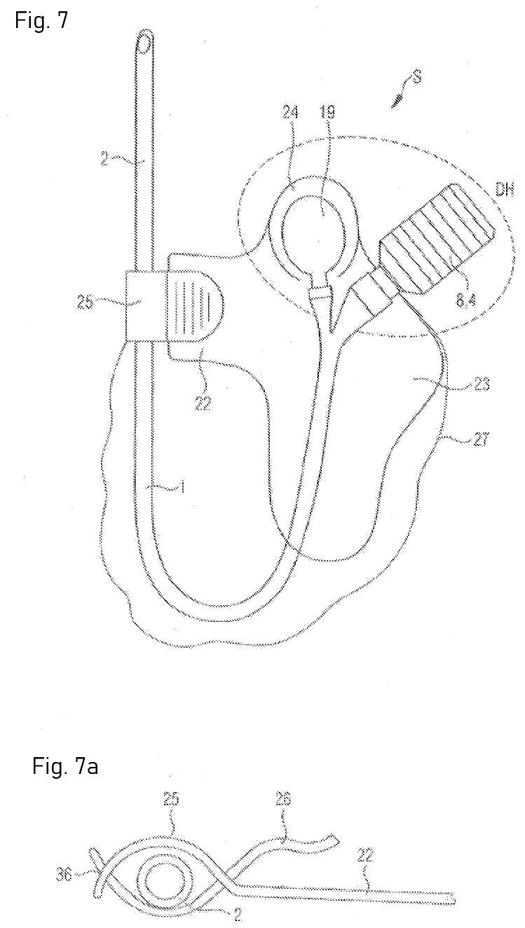

FIG. 7. The depicted U-shaped configuration of the catheter set device S shown here, consisting of a urinary catheter 1 according to the invention, an insertion aid 4 that is inserted into the draining lumen of the catheter, and connected reservoir vessels 8, 19, is ensured by a holding device 22 adapted in terms of its shape to the palm of the user's hand, which, on the one hand, accommodates and fastens the proximal, connector-side end of the catheter, and, on the other hand, grips the distal shaft portion of the catheter. The holding device optionally has a palmar handle piece 23 for grasping the holding device in a safe manner. The thumb-side portion of the holding device DH accommodates the proximal part of the catheter arrangement. In a preferred design, the connector parts of the catheter arrangement are fastened in custom-fit preformations of the handle piece or of the holding device in a locking manner for example. The fastening thereby makes an adequate abutment function possible, so that gel can be conveyed towards the catheter tip, for example via thumb pressure on the base of the reservoir without causing a detachment of the connector parts from the fastening due to the applied pressure of the thumb. On the other hand, after placement of the catheter or the securing thereof in the bladder, the entire proximal connector part can be removed from the locking position by the user with reasonable resistance. C-shaped deep-drawn parts accommodating the catheter shaft and/or the connector parts are advantageous here. In the process, the respective portions of the device engage in a preferably elastically deformable holding clasp. Because of a convex deformation or curvature of the holding device, for example due to squeezing the holding clasp in the palmar hand, the C-shaped profile of the deep-drawn part is opened, and the catheter is thus released.

In particular the gel vessel 8 is positioned in the clasp or in the handle piece in such a way that it can be condensed or compressed easily by the user. The filling vessel for blocking the balloon can be arranged adjacent to the gel vessel. It can be compressed or squeezed in an appropriate manner by thumb pressure. A balloon-like design that is filled with air can be embedded for example in a spoon-like depression 24 of the clasp or of the handle and rest there virtually on an abutment.

To ensure that the catheter is protected from contamination during placement of the catheter, the holding device is equipped with a thin-walled, bag-like film 27. In the process, the bag film protects in particular the catheter section, which extends from the gripping tweezers or tongs 25 to the connector of the catheter device. The protecting bag is designed for accommodating the looped, centric catheter shaft. The film protection makes it possible for the user to dispense with putting on sterile gloves and insert the catheter equipped or fastened in the holding device according to the invention in a sterile manner.

FIG. 7a describes a special, tweezer-like or tong-like clamp 25 that is attached laterally to the holding device 22. If the rocker 26 is loaded with thumb pressure, the clamp grips the catheter shaft 2 that is to be inserted. If the rocker is released, the clamp is displaced into a new relative position with respect to the catheter shaft. The rocker-like or tong-like clamp is preferably designed in such a way that the gripping elements are claw-like and the claws thereby comprise a certain overhang 36, which, in an unloaded state of the tongs, makes the catheter axially movable namely within the claw, but prevents the complete release of the catheter from the holding device, and only makes it possible with actuation of the rocker in the opposite direction.

Once the roughly first ten centimeters of the catheter shaft, which are moistened with a lubricating substance, are inserted into the urethra, the catheter shaft in the clamp is released by releasing the rocker, and it can then be displaced proximally towards the shaft, where the clamp again grips the shaft in the case of pressure on the rocker. The catheter shaft can thus be pushed forward by the user successively into the urethra in the manner of a tweezer-like of tong-like guidance. Once roughly two thirds of the shaft length is inserted into the urethra, the catheter is secured by filling the balloon, and the catheter is removed completely from the fastening deep-drawn parts or formations of the holding device.

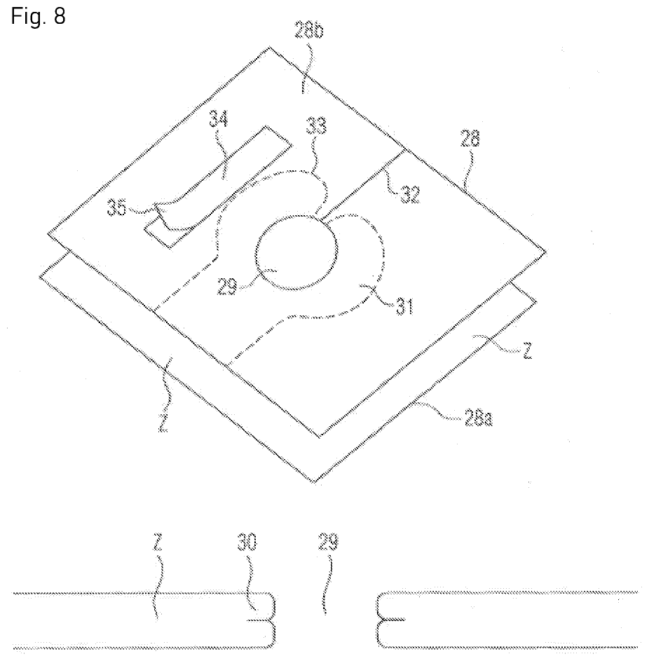

FIG. 8 shows a special design of a surgical drape 28, which is made of two layers of a preferably non-woven-like material, wherein the two layers are connected to each other in a tightly sealing manner in the region of the centrally arranged hole 29 via a seam 30 by means of bonding or fusing. The user picks up the drape, in that he or she reaches into the intermediate space Z between the two layers with the hand that is opened in a C-shaped manner and finally grips around the hole 29. The direct control of the hole by gripping around it in a C-shaped manner with the thumb and the index finger allows the opening to be guided in an optimally formed manner to the to-be-cleaned surfaces surrounding the orifice or to grip the penis optimally in an supportive manner or to spread open the labia. As an alternative to an intermediate space Z, the surgical drape 28 can be equipped on the side 28a facing the patient with a generously dimensioned glove-like element 31 that is customized in a stylized manner, the rear side of which is connected in a planar manner to the side 28a facing the patient. In another less preferred design variant, the element 31 on the palmar side can be connected to the side 28b of the surgical drape that faces away from the patient. The surgical drape can be provided with a continuous slit 32 originating from the hole and going towards the periphery of the drape. Furthermore, the drape can be equipped with blister-pack-like compartments 34 preformed for the individual accommodation of saturated swabs, arranged preferably in a line above the ulnar edge 33 of the hand that is picking up the surgical drape. The blister pack is sealed off from the user with a film 35 that can be torn off. The user grips the individual swabs at a stamp-like extension enclosed in protective film and removes them from the blister pack in this manner.

The invention moreover describes a method for catheterization the bladder of a patient, wherein the number of components required for the cleaning and insertion process is kept to a minimum. The described ready-to-use, prepared catheter device according to the invention is advantageously combined for this purpose with a surgical drape 28 designed in a glove-like manner to directly grip the penis or spread open the labia, which surgical drape, like the catheter device, is designed in a special manner for a one-handed handling. In conjunction with the two functional units (glove/drape and catheter device), the user is able to carry out the entire catheterization process without a person assisting them.

The two functional units are packaged together in a set. After opening the outer packaging of the set, the user uses their hand to grip directly into the surgical drape unit that is designed in a glove-like manner and thereby take it out of the set. The user then directly grips the penis or spreads open the labia. They thereby achieve an optimally protective position of the drape vis-a-vis the orifice to be cleaned and then catheterized. Then they fold the lateral portions of the drape as needed and fasten them to the abdomen, leg or on the buttocks of the patient using the corresponding self-adhesive surfaces.

As a special feature, the glove/drape 28 optionally comprises blister-pack-like, deep-drawn film compartments 34 that are integrated into the drape, and are opened by tearing open or peeling off a protective film 35 with the free hand, and contain ready-to-use swabs that are saturated with antiseptic for cleaning the skin and mucous membranes. The swabs are picked out the blister pack at a stamp-grip-like formation, which is protected by the sealed deep-drawn film and thereby allows the user to grip in a direct and contamination-free manner. The swabs can then be discarded on the surgical drape or in a pocket-like formation attached thereto.

After cleaning has been carried out, the user then grips the catheter device with their free hand and inserts the distal catheter portion that is lubricated with gel and stabilized by the insertion aid, reinforced by the insertion aid and guided by the holding device in a lance-like manner into the urethra. Using thumb pressure, they then apply the substance still remaining in the reservoir vessel into the urethra and move the catheter by successive gripping and releasing of the catheter shaft with the clamp of the holding device into the bladder. During insertion of the catheter, the user adjusts the penis into the respective optimal position via the surgical drape using a direct grip or they keep the labia optimally open by digital spreading.

Once the first two thirds of the catheter are inserted into the urethra, the balloon of the catheter is blocked by pressing the filling vessel with the thumb. Then, the hand is removed from glove/drape combination and the proximal catheter portion is retracted from the holding device.

The insertion aid is then retracted from the device and the filling vessel is removed from the catheter valve. The catheter is then connected to a urine-draining hose/bag unit.

The retaining balloon component of the urinary catheter is preferably formed by blow molding from a polyurethane tube blank that was previously blow molded in a separate manufacturing step, preferably in a durometer range of Shore 80A to 95A, especially preferably between 85A and 90A. The wall thickness is preferably 5 to 25 μm, especially preferably 7 to 12 μm. When using material types such as for example PUR of the Elastollan 1100 group from BASF or PUR of the Pellethane 2363 type from Lubrizol, flexible film-like structures can also be blown out of pre-extruded material using blow molding, which structures connect the lowest wall thickness with high dimensional stability in an ideal way. Particularly in the case of a high axially directed outward tensile force effect, micro-thin PUR balloon components ensure a reliable anchoring effect.

In addition to polyurethane, the balloon element can also be fabricated less preferably of comparable flexible films of alternative materials, such as for example polyethylene-based mixtures or TPE-based raw materials. Furthermore, co-extruded starting materials that combine for example PUR and PVC in a coaxially extruded manner in the raw tube are conceivable for the blow molding.

Likewise less preferred, it is also possible to use conventional sleeve-based, non-preformed balloon elements that are to be elastically expanded.

List of Reference Numbers

| | |
|---|---|
| 1 | Device |
| 2 | Indwelling part, catheter |
| 2a | Gap space |
| 3 | Balloon |
| 4 | Insertion aid |
| 5 | Tip |
| 6 | Drainage opening |
| 7 | Opening |
| 7a | Tip, tip formation |
| 7b | Bulge, thickening |
| 7c | Corrugated profile |
| 8 | Reservoir vessel |
| 8a | Reservoir unit |
| 9 | Segment |
| 9a | Portion |
| 9b | Funnel-like formation |
| 9c | Perforable surface |
| 9d | Thorn-like extension |
| 10 | Connector |
| 11 | Shaft |
| 12 | Lumen |
| 13 | Section, profile |
| 14 | Shaft tube |
| 15 | Boden |
| 16 | Segment, region |
| 17 | Element, closure |
| 18 | Hood-like element |
| 19 | Bellows, vessel |
| 20 | Colored marking |
| 21 | Connection cone |
| 22 | Holding device |
| 23 | Handle piece |
| 24 | Spoon-like depression |
| 25 | Tong, clamp |
| 26 | Rocker |
| 27 | Protective pouch |
| 28 | Surgical drape |
| 28a | Side facing the patient |
| 28b | Side facing away from the patient |
| 29 | Hole |
| 30 | Seam |
| 31 | Element |
| 32 | Slit |
| 33 | Edge |
| 34 | Film compartment |
| 35 | Film, protective film |
| 36 | Overhang |
| DH | Thumb-side holding part |
| KO | Connector part |
| P | Laterally bent position |
| S | Set, catheter set |
| SR | Gap space |
| SE1 | Rod-shaped element |
| SE2 | Tubular element |
| VS | Intravesical segment |
| Z | Intermediate space |

1. A device (1) for draining urine from the bladder of a patient, comprising
a) a shaft-shaped indwelling part (2) that can be placed in the bladder and in the urethra that drains in the manner of a catheter and has at least one drainage opening (6) in the region of the distal tip (5) of the shaft-shaped indwelling part (2); and
b) a retractable insertion aid (4) that is inserted into the shaft-shaped indwelling part (2), which facilitates insertion into and passage through the urethra, and has a tubular or hose-like construction with an apical opening (7) for terminal release of a lubricating and/or lumen-straightening substance through the opening (7).

2. The device (1) according to claim 1, characterized in that the substance can be applied in a sealing manner from the apical opening (7) of the insertion aid (4) through a draining opening (6) in the region of the catheter tip in such a way that a reverse flow of the substance into the gap area (2a) between the insertion aid and the draining catheter lumen, which gap area is proximally adjacent at the opening of the insertion aid, is reliably prevented.

3. The device (1) according to claim 1, characterized in that the distal opening (7) of the insertion aid (4), is directly adjacent to the distal, draining opening (6) of the indwelling part (2), into which the draining lumen of the indwelling part (2) opens or is located in a direct covering or common alignment with the draining opening (6) of the indwelling part (2).

4. The device (1) according to claim 1, characterized in that the tip (7a) of the insertion aid (4) extends beyond the distal, draining opening (6) of the indwelling part (2).

5. The device (1) according to claim 4, characterized in that the tip (7a) of the insertion aid (4)
a) comprises a curved preformation for facilitating passage through the urethra, and/or
b) is preformed, in particular in a corrugated manner, for an increased flexibility, in order to make a flexible bending of the tip (7a) of the insertion aid (4) possible, which bendability preferably reaches beyond 90 degrees, or in an ideal case beyond 135 degrees.

6. The device (1) according to claim 4, characterized in that the tip (7a) of the insertion aid (4) in the transition region to the distal opening (6) of the indwelling part (2) comprises a bulge-shaped, bulb-shaped or spindle-shaped cross-section expansion (7b) smoothing the transition, which cross-section expansion deforms from the proximal side in such a way under the effect of tensile force that the cross-section expansion (7b) can be pulled out through the lumen of the preferably catheter-shaped indwelling part (2).

7. The device (1) according to claim 1, characterized in that, in the region of a connector (10) of the urinary catheter or of the indwelling part (2), the insertion aid (4) can be secured in terms of its position by slight pressing or locking in its position, and thereby comprises a region (9) formed with a custom-fit and congruent shape.

8. The device (1) according to claim 1, characterized in that the distal region of the insertion aid (4) is made of a tubular or hose-like extension, optionally reinforcing the device (1), from which a substance-accommodating reservoir unit (8) in a one-piece design preferably directly emerges.

9. The device (1) according to claim 1, characterized in that the proximal region of the insertion aid (4) comprises a funnel-like formation (9b), into which a structurally separate reservoir unit (8, 8a) can be inserted in a tightly sealing manner via a portion (9a) that is formed with a congruent shape.

10. The device (1) according to claim 9, characterized in that the inserted reservoir unit (8, 8a) comprises on the distal end a perforable surface (9c), which can be opened by a thorn-like extension (9d) of the distal region of the insertion aid (4) in the case of complete, terminal insertion of the reservoir (8, 8a).

11. The device (1) according to claim 1 onc of thc prcccding claims, characterized in that the reservoir unit (8, 8a) is made of a material formed to be bellows-like, which during compression of the bellows is preferably fastened in the respective position in a locking manner and prevents a reestablishing of the bellows.

12. The device (1) according to claim 11, characterized in that the reservoir unit (8, 8a)
a) comprises segments (16) marked in color, whereby during the engaging compression thereof can code the release of a specific volume, and/or
b) comprises segments (16), which during manual compression develop respectively specific resistances to folding and/or collapse, and thus facilitate a tactilely perceptible transition from a first volume quantity to be dosed to a subsequent second volume quantity.

13. The device (1) according to claim 1, characterized in that the shaft portion of the indwelling part (2) in one or more sections (13) of the shaft is preformed in a corrugated manner, whereby the axial bending of the shaft is facilitated and elastically acting restoring forces can be reduced in the shaft.

14. The device (1) according to claim 1, characterized in that the shaft portion in the urethral section or in the section between the retention balloon (3) and the proximal connector (10) of the indwelling part (2) is made of film-like, thin-walled tube material (14).

15. The device (1) according to claim 14, characterized by one or more rod-like or tubular structure(s) (SE1) arranged preferably exposed in the inner lumen of the tubular film (14), in particular in order to prevent the occlusion of the drainage lumen as a result of axial twisting or kinks of the tubular film (14).

16. The device (1) according to claim 14, characterized by a net-like reinforcement (SE2) that is permanently connected to the tubular film (14) or integrated therein, which reinforcement elastically straightens the lumen of the tubular film (14), in particular in order to hinder axial twisting and/or return any twisting that has occurred back into the non-torqued starting position.

17. The device (1) according to claim 1, characterized in that the distal region of the indwelling part (2) and/or of the insertion aid (4) is provided with a hood-like element (18).

18. The device (1) according to claim 17, characterized in that the gap space (SR) between the hood (18) and the indwelling part (2) can be filled by in particular a lubricating and/or lumen-straightening substance that is fed into the gap space (SR).

19. The device (1) according to claim 18, characterized in that the hood (18) apically comprises for example a tap-like closure element (17), which is permanently connected to the hood (18), and which seals the outlet opening of the insertion aid (4) in a plug-like manner, and which, after disengagement thereof from the insertion aid (4), facilitates the release of the lubricating and/or lumen-straightening substance from the inner lumen of the insertion aid (4) along with the terminal, reservoir-like expansion (8, 8a) thereof.

20. The device (1) according to claim 1, characterized in that the unit, which consists of the indwelling part (2) and an insertion aid (4) inserted into the indwelling part (2), is inserted in a holding device (23, 25) and is reversibly fastened there, and in the process is put into a U-shaped, sling-like configuration, wherein the distal part of the unit (2, 4) is held by a tong-like or rocker-like opening and closing device (25), and the proximal region of the unit (2, 4) bearing the connector (10) is accommodated in the holding device (25) in the preferably C-shaped recesses in such a way that a filling bellows or filling vessel (19) inserted or plugged into the connector part (10) and/or a reservoir vessel (8, 8a) possibly inserted or plugged in there can be compressed or emptied by thumb pressure.

21. The device (1) according to claim 20, characterized in that the proximal region of the indwelling part (2), preferably approximately the proximal half of the indwelling part (2), in particular approximately the proximal two thirds of the indwelling part (2), is accommodated in a surrounding film-like protective pouch (27), which is preferably connected permanently or even detachably to the holding device (23, 25) that is accommodating the indwelling part (2).

22. The device (1) according to claim 20, characterized in that the tong-like or rocker-like device (25) facilitates an overbite-like gripping (36) of the shaft of the indwelling part (2) in the opening lateral part, which, when disengaging the indwelling part (2), prevents the shaft (2) of the indwelling part (2) from being able to move out of the grip of the tongs (25) and thus be released from the holding device (25), and the overbite (36) thereof first opens in the case of a maximum opening of the rocker (26) and thereby releases the catheter shaft (2).

23. The device (1) according to claim 1, characterized in that the material of the indwelling part (2) comprises a Shore hardness (x), and that the material of the insertion aid (4) comprises a Shore hardness (y), wherein the Shore hardness (y) of the material of the insertion aid (4) corresponds to or exceeds that of the Shore hardness (x) of the material of the indwelling part (2), in other words:
y=x
or
y>x.

24. A method for catheterization of the bladder of a patient using a ready-to-use, prepared catheter device, comprising a shaft-shaped indwelling part (2) that can be placed in the bladder and in the urethra that drains in the manner of a catheter and has a drainage opening (6) in the region of the distal tip (5) of the shaft (2), and a retractable insertion aid (4) that is inserted into the indwelling part (2), which facilitates insertion into and passage through the urethra, and has a tubular or hose-like construction with an apical opening (7) for terminal release of a lubricating and/or lumen-straightening substance, characterized in that the lubricating and/or lumen-straightening substance is released from a compressible reservoir vessel (8, 8a), which is or will be attached at the proximal end of the insertion aid (4), as a result of a digital or manual compression of the reservoir vessel (8, 8a).

25. The method according to claim 24, characterized in that the lubricating and/or lumen-straightening substance is released or injected at least partially into a gap space (SR) between the indwelling part (2) and a hood (18) externally surrounding the distal region thereof.

26. The method according to claim 25, characterized in that, after the at least partial filling of the gap space (SR) between the indwelling part (2) and the hood (18), the hood (18) is pulled off of the indwelling part (2).

27. The method according to claim 24, characterized in that the externally lubricated, distal end of the indwelling part (2) is inserted into the urethra.

28. The method according to claim 24, characterized in that, after inserting the distal end of the indwelling part (2) into the urethra, a possibly additional quantity of the lubricating and/or lumen-straightening substance is released or injected into the urethra in order to open the urethra for the further feed of the indwelling part (2).

29. The method according to claim 28, characterized in that, after insertion into the urethra, the indwelling part (2) is fixed at the desired position, in that a balloon (3) arranged externally on the shaft of the indwelling part (2) preferably in the region of the distal end thereof is filled with a medium, preferably with air.

30. The method according to claim 24, characterized in that, after positioning the indwelling part (2) in the urethra and/or after fixing same at the desired position, the insertion aid (4) is removed from the indwelling part (2).

31. The method according to claim 24, characterized in that, after removing the insertion aid (4) from the indwelling part (2), a connector part (KO) for a collection bag or the like is attached to the proximal end of the indwelling part (2).

32. The method according to claim 24, characterized in that the indwelling unit (2), as the case may be, with a balloon (3) arranged on the outer circumference thereof, the insertion aid (4), the reservoir vessel (8, 8a) with a filling of the lubricating and/or lumen-straightening substance, as the case may be, a tong-shaped holding device (25) along with, as the case may be, a manual apparatus for filling the balloon (3) of a preferably common protective pouch (27) are removed, where they are held in readiness as the catheter device.

33. The method according to claim 24, characterized in that, before the insertion of the indwelling part (2) into the urethra, the surrounding area of the urethral opening is covered by a surgical drape (28).

34. The method according to claim 33, characterized in that a surgical drape (28) is used, which is designed in a glove-like manner and when put on via one hand, allows the penis to be gripped directly or the labia to be spread open.

35. The method according to claim 33, characterized in that, prior to the catheterization process, a swab is removed from an initially sealed compartment of the surgical drape unit (28), and the urethral opening and/or the surrounding area thereof is disinfected with it.

36. The method according to claim 24, characterized in that the catheterization process is carried out without a person assisting, in that both the catheter device and the glove/surgical drape (28) are respectively designed as functional units for a one-handed handling.

37. The method according to claim 36, characterized in that both functional units are packaged together in a set, so that, after opening the outer packaging of the set, the user is able to grip with one hand directly into the surgical drape unit (28) that is designed in a glove-like manner in order to thereby take it out of the set.

38. The method according to claim 36, characterized in that, after removing the surgical drape unit (28) from the outer packaging, the user directly grips the penis or spreads open the labia, then they pick up the lateral portions of the drape as needed and fasten them to the abdomen, leg or on the buttocks of the patient, preferably using the corresponding self-adhesive surfaces.

39. The method according to claim 36, characterized in that, after applying the surgical drape unit (28) to the patient, the user removes at least one ready-to-use swab, which is saturated with antiseptic, from optionally blister-pack-like, deep-drawn film compartments (34) that are integrated into the glove/drape (29) and are opened by tearing open or peeling off a protective film (35) with the free hand, and uses said swab to clean the skin and/or mucous membrane.

40. The method according to claim 36, characterized in that, after cleaning has been carried out, the user grips the ready-to-use catheter device with their still free hand and inserts the distal catheter portion that is lubricated with gel and stabilized by the insertion aid (4) guided in a lance-like manner into the urethra.

41. The method according to claim 36, characterized in that, after inserting the distal catheter portion into the urethra, the user applies the substance still remaining in the reservoir vessel (8) into the urethra via thumb pressure.

42. The method according to claim 36, characterized in that, after applying the substance in the urethra, the user moves the catheter forward into the bladder by successive gripping and releasing of the catheter shaft with the clamp of the holding device (25), preferably wherein, during the insertion of the catheter, they position the penis using a direct grip with the surgical drape (28) in the respective optimal position for the catheterization or keep the labia open by spreading.

43. The method according to claim 36, characterized in that, after inserting the distal region of the indwelling unit (2), in particular the first two thirds of the catheter (2), into the urethra, the user fills the balloon (3) of the catheter (2) by pressing the filling vessel (19) with their thumb or locks the position thereof.

44. The method according to claim 43, characterized in that, after filling or locking the balloon (3), the user removes their hand from the glove/drape combination (28) and retracts the proximal catheter portion from the holding device.

45. The method according to claim 44, characterized in that, after retracting the proximal catheter portion from the holding device, the user retracts the insertion aid (4) from the urinary catheter (2) and disconnects the filling vessel (19) from the catheter valve.

46. The method according to claim 45, characterized in that, after disconnecting the filling vessel from the catheter valve, the user connects the catheter to a urine-draining hose/bag unit.
